(19) United States
(12) Patent Application Publication
(10) Pub. No.: US 2015/0079118 A1
(43) Pub. Date: Mar. 19, 2015

(54) MicroRNA Treatment for Asthma

(71) Applicant: UNIVERSITY OF SOUTHAMPTON, Southampton (GB)

(72) Inventors: Peter Hugo HOWARTH, Bransgore (GB); Tilman SANCHEZ-ELSNER, Southampton (GB); Hitasha RUPANI, Southampton (GB)

(21) Appl. No.: 14/490,280

(22) Filed: Sep. 18, 2014

(30) Foreign Application Priority Data
Sep. 19, 2013 (GB) 1316651.7

(51) Int. Cl. C12N 15/113

(52) U.S. Cl. CPC C12N 15/113; C12N 2310/17; C12N 2310/113; C12N 2320/31
USPC 424/184.1

(57) ABSTRACT

This invention relates to a composition for modifying the activity of microRNA-mediated regulation of mRNA in a cell, wherein the composition comprises: two or more compounds capable of blocking two or more microRNA molecules from binding to the same target mRNA, wherein the two or more microRNA molecules are different in sequence or structure relative to each other. In particular, compositions for use in the treatment of asthma and methods of prevention or treatment of asthma.

This invention relates to a composition for modifying the activity of microRNA-mediated regulation of mRNA in a cell. In particular, compositions for use in the treatment of asthma and methods of prevention or treatment of asthma.

MicroRNAs, also referred to herein as miRNAs, are a class of small endogenously expressed small regulatory non-coding RNAs of about 18 to 25 nucleotides in length. miRNAs negatively regulate target mRNAs. This negative regulation is mediated by binding to a miRNA binding site which is an imperfect complement to the miRNA in the 3′-untranslated region of the mRNA. These are also referred to herein as 3′-UTRs. The miRNA can alternatively bind in the coding sequence of the mRNA. MicroRNAs play a crucial role in the complex network of gene regulation in eukaryotic cells. As they are the specificity determining subunit in the RNA-induced silencing complex (RISC), miRNAs are capable of inhibiting the translation of genes. Based on early studies in invertebrates, miRNAs are expected to have roles in developmental regulation and cell differentiation in mammals, and roles for miRNAs in cardiogenesis and lymphocyte development have been demonstrated. Several studies suggest a strong connection between miRNA and human cancer. Recent reports implicate roles for mammalian miRNAs in metabolic pathways. Furthermore, miRNAs may contribute to destabilization of the mRNA molecules bound by the miRNA. Despite the growing list of roles for mammalian miRNAs, most of the hundreds of miRNAs identified in mammals have no reported function. Nevertheless, many miRNAs and their respective targets have been found to be evolutionary highly conserved. One single miRNA may regulate a variety of different genes, in some examples said variety consisting of more than 100 genes. Accordingly, it is generally assumed today that between 30 and 60% of all human genes are regulated by miRNAs.

MicroRNAs have also been shown to suppress levels of viral RNA in cells. In mammals, miRNAs can play diverse roles in viral infection through their capacity to regulate both host and viral genes. Recent reports have demonstrated that specific miRNAs change in expression level upon infection and can impact viral production and infectivity. It is clear that miRNAs are an integral component of viral-host interactions, and it is likely that both host and virus contain mechanisms to regulate miRNA expression and/or activity.

Asthma is one of the most common chronic diseases globally, with recurrent exacerbations and resistance to treatment significantly contributing to the morbidity and economic burden of this airway disorder. Up to 85% of exacerbations are caused by respiratory viruses, and asthmatics show increased susceptibility to viral infection. Severe asthma sufferers, even on maximal therapy, experience recurrent exacerbations and thus it is vital that the mechanisms underlying the susceptibility to virally triggered exacerbations are elucidated in order to develop preventive treatment strategies. Bronchial epithelial cells and cells recovered by bronchoalveolar lavage (BAL) in asthmatics appear to have an impaired innate immune response to viruses, producing less interferon (IFN) than cells from healthy subjects. Among the cells recovered in BAL, alveolar macrophages (AM) are key to airway defences, promoting airway tolerance to antigens as well as detecting invading viruses with the consequent secretion of IFN that protects airway epithelium from rhinoviruses. Many aspects of innate immune functions and signalling pathways appear to be controlled by microRNAs (miRNAs). miRNAs are small non-coding RNA molecules that exert post-transcriptional regulation on gene expression by repression of translation or degradation of mRNA.

Therefore, it would be desirable to provide methods and compositions to bring the airway reactivity of asthmatics to viruses and bacteria closer to that of normal subjects.

According to a first aspect of the invention, there is provided a composition for modifying the activity of microRNA-mediated regulation of mRNA in a cell, wherein the composition comprises:
two or more compounds capable of blocking two or more microRNA molecules from binding to the same target mRNA,
wherein the two or more microRNA molecules are different in sequence or structure relative to each other.

MicroRNAs are small non-coding RNAs that inhibit gene expression by pairing to the 3′ untranslated region (3′UTR) of their target mRNAs facilitating their translational repression or degradation. It has been found that two or more micro RNA molecules may target the 3′untranslated region of the same mRNA. MicroRNA molecules that have a specific target mRNA in common tend to increase their affinity for this target when they are simultaneously manipulated in the cell. Having an increased preference for the common target, these microRNA molecules seem to show less avidity for other targets, thereby reducing the potential for off-target effects of a potential microRNA regulation based therapy. In particular, microRNAs target a region in the mRNA, the 3′UTR (untranslated region) that is in a tight secondary structure (this is difficult to access). MicroRNAs targeting areas that are slightly open are more effective. Thus, more than one microRNA targeting the same mRNA will make it more accessible to the rest (they will co-operate to open this closed region). Not only will this increase their effect on this particular mRNA, but it will favour the mRNA targeted by several microRNAs (because it is open and accessible) over the other targets that are still closed and are difficult to access, which reduces the chance of off target effects. Therefore the advantage of targeting multiple microRNA molecules is that they may eliciting a stronger and/or more controlled response than targeting individual microRNA molecules. It is advantageous to simultaneously or sequentially administer compounds that bind to each of a group of microRNA molecules that all target the same mRNA to provide an enhanced effect on the mRNA.

Advantageously, blocking the binding of microRNA molecules to mRNA can elevate the mRNA levels in the cell (thereby upregulating the expression of the encoded protein), because binding of the microRNA molecules to the mRNA can mark the mRNA for degradation or block their translation into protein.

The cell may be a mammalian cell. The cell may be a human cell. The cell may be an alveolar macrophage. The cell may be human alveolar macrophage.

At least one microRNA molecule may be selected from any of the group comprising miR-150, miR-152 and miR-375. The two or more microRNA molecules may comprise two or more microRNA molecules selected from any of the group comprising miR-150, miR-152 and miR-375. The two or more microRNA molecules may comprise three or more microRNA molecules selected from any of the group comprising miR-150, miR-152 and miR-375. The two or more microRNA molecules may comprise miR-150 and miR-152. The two or more microRNA molecules may comprise miR-150 and miR-375. The two or more microRNA molecules may comprise miR-152 and miR-375. The two or more microRNA molecules may comprise miR 150, miR-152 and miR-375.

The mRNA may encode TLR7. The mRNA may encode RipK1. The microRNA molecules may target the mRNA encoding TLR7 and RipK1.

At least one compound may be arranged to bind to a microRNA molecule to prevent it from engaging with the target mRNA. Two or more compounds may be arranged to bind to the microRNA molecules to prevent them from engaging with the target mRNA. At least one compound may be arranged to bind to the target mRNA in order to block a binding site of a microRNA molecule. Two or more compounds may be arranged to bind to the target mRNA in order to block a binding site of the microRNA molecules.

The compound may be an antagomir, oligonucleotide, or blockmir. At least one compound may be an oligonuleotide. At least one compound may be an antagomir. At least one compound may be a blockmir. A combination of antagomirs and blockmirs may be provided. The two or more compounds may be selected from the group comprising oligonuleotides, antagomirs and blockmirs, or combinations thereof.

The compound may be the Anti-miR™ miRNA inhibitor of miR-150 or mirVana® miRNA inhibitor of miR-150 (available from Life Technologies Corporation Cat. #AM17000 and Cat. #4464084 respectively) The compound may be the Ambion® Anti-mir™ miRNA inhibitor of miR-150 (available from Life Technologies Corporation). The compound may be an antagomir having at least about 80% sequence identity to the Anti-miR™ miRNA inhibitor of miR-150; or mirVana® miRNA inhibitor of miR-150; or Ambion® Anti-mir™ miRNA inhibitor of miR-150. The compound may be an antagomir having at least about 90%, 95%, 98%, or 99% sequence identity to the Anti-miR™ miRNA inhibitor of miR-150 or mirVana® miRNA inhibitor of miR-150; or Ambion® Anti-mir™ miRNA inhibitor of miR-150.

The compound may be the Anti-miR™ miRNA inhibitor of miR-152 or mirVana® miRNA inhibitor of miR-152 (available from Life Technologies Corporation Cat. #AM17000 and Cat. #4464084 respectively). The compound may be the Ambion® Anti-mir™ miRNA inhibitor of miR-152 (available from Life Technologies Corporation). The compound may be an antagomir having at least about 80% sequence identity to the Anti-miR™ miRNA inhibitor of miR-152; or mirVana® miRNA inhibitor of miR-152; or Ambion® Anti-mir™ miRNA inhibitor of miR-152. The compound may be an antagomir having at least about 90%, 95%, 98%, or 99% sequence identity to the Anti-miR™ miRNA inhibitor of miR-152; or mirVana® miRNA inhibitor of miR-152; or Ambion® Anti-mir™ miRNA inhibitor of miR-152.

The compound may be the Anti-miR™ miRNA inhibitor of miR-375 or mirVana® miRNA inhibitor of miR-375 (available from Life Technologies Corporation Cat. #AM17000 and Cat. #4464084 respectively). The compound may be the Ambion® Anti-mir™ miRNA inhibitor of miR-375 (available from Life Technologies Corporation). The compound may be an antagomir having at least about 80% sequence identity to the Anti-miR™ miRNA inhibitor of miR-375; or mirVana® miRNA inhibitor of miR-375; or Ambion® Anti-mir™ miRNA inhibitor of miR-375. The compound may be an antagomir having at least about 90%, 95%, 98%, or 99% sequence identity to the Anti-miR™ miRNA inhibitor of miR-375; or mirVana® miRNA inhibitor of miR-375; or Ambion® Anti-mir™ miRNA inhibitor of miR-375.

At least one compound may be an oligonuleotide comprising a sequence complementary to a sequence selected from any of the group comprising SEQ ID NO. 24, SEQ ID NO. 25, and SEQ ID NO. 26. The sequence may be complementary across between about 5 and 15 nucleotides, or 6 and 10 nucleotides. The sequence may be complementary across between 7 and 9 nucleotides.

The two or more compounds may comprise two or more oligonuleotides comprising a sequence complementary to a sequence selected from any of the group comprising SEQ ID NO. 24, SEQ ID NO. 25, and SEQ ID NO. 26. The two or more compounds may comprise three or more oligonuleotides comprising a sequence complementary to a sequence selected from any of the group comprising SEQ ID NO. 24, SEQ ID NO. 25, and SEQ ID NO. 26. The two or more compounds may comprise an oligonuleotide comprising a sequence complementary to a sequence of SEQ ID NO. 24 and an oligonucleotide comprising a sequence complementary to a sequence of SEQ ID NO. 25. The two or more compounds may comprise an oligonuleotide comprising a sequence complementary to a sequence of SEQ ID NO. 24 and an oligonucleotide comprising a sequence complementary to a sequence of SEQ ID NO. 26. The two or more compounds may comprise an oligonuleotide comprising a sequence complementary to a sequence of SEQ ID NO. 25 and an oligonucleotide comprising a sequence complementary to a sequence of SEQ ID NO. 26. The two or more compounds may comprise an oligonuleotide comprising a sequence complementary to a sequence of SEQ ID NO. 24; an oligonucleotide comprising a sequence complementary to a sequence of SEQ ID NO. 25; and an oligonucleotide comprising a sequence complementary to a sequence of SEQ ID NO. 26.

At least one compound may be an oligonuleotide comprising a sequence complementary to a sequence selected from any of the group comprising SEQ ID NO. 24, or complementary to a sequence having 80%, 90%, 95%, or 99% identity to SEQ ID NO. 24. At least one compound may be an oligonuleotide comprising a sequence complementary to a sequence selected from any of the group comprising SEQ ID NO. 25, or complementary to a sequence having 80%, 90%, 95%, or 99% identity to SEQ ID NO. 25. At least one compound may be an oligonuleotide comprising a sequence complementary to a sequence selected from any of the group comprising SEQ ID NO. 26, or complementary to a sequence having 80%, 90%, 95%, or 99% identity to SEQ ID NO. 26.

The sequence may be complementary across at least 5 nucleotides. The sequence may be complementary across at least 6 nucleotides, at least 7 nucleotides, at least 8 nucleotides, at least 9 nucleotides, or at least 10 nucleotides.

One or more compounds may be anti-microRNA oligonucleotides that specifically bind to the microRNA molecules. The oligonucleotides may have more than 60%, more than 70%, more than 80%, more than 90%, more than 95%, more than 98%, more than 99% or 100% sequence identity to a sequence that is complementary to the microRNA molecule that it specifically binds to. The sequence identity may be defined over the entire region of the anti-microRNA oligonucleotide.

One or more compounds may be oligonucleotides that specifically bind to the microRNA molecule binding site of the target mRNA. The oligonucleotides may have more than 60%, more than 70%, more than 80%, more than 90%, more than 95%, more than 98%, more than 99% or 100% sequence identity to a sequence that is complementary to the microRNA molecule binding site of the mRNA that it specifically binds to.

The sequences identities may be determined by BLAST under standard parameters.

One or more compounds may be anti-microRNA oligonucleotides that are long enough to specifically bind to their target microRNA and block or reduce their binding to their target mRNA. The anti-microRNA oligonucleotides may be between 5 and 50 nucleotides in length, between 10 and 40 nucleotides in length, between 15 and 30 nucleotides in length, or between 20 and 25 nucleotides in length. The anti-microRNA oligonucleotides may be between 7 and 9 nucleotides in length.

One or more compounds may be oligonucleotides that are long enough to specifically bind to the microRNA binding site of the mRNA and block or reduce the binding of the microRNA molecules to the target mRNA. The oligonucleotides may be between 5 and 50 nucleotides in length, between 10 and 40 nucleotides in length, between 15 and 30 nucleotides in length, or between 20 and 25 nucleotides in length.

At least one compound may be a small molecule or a peptide. The two or more compounds may be small molecules or peptides.

A small molecule may be a chemical compound. A small molecule may not be a biological molecule. The small molecule may not be any one of a polymeric nucleic acid, a protein, or an antibody. A small molecule may be a low molecular weight <900 Daltons, organic compound that may serve as an enzyme substrate or regulator of biological processes, with a size on the order of 10⁻⁹ m. Biopolymers such as nucleic acids, proteins, and polysaccharides (such as starch or cellulose) may not be considered small molecules. The constituent monomers of biopolymers, such as ribo- or deoxyribonucleotides, amino acids, and monosaccharides may be considered to be small molecules. Small oligomers may be considered small molecules, such as dinucleotides, peptides such as the antioxidant glutathione, and disaccharides such as sucrose.

The composition may comprise three or more different compounds wherein each of the three or more different compounds binds to a different microRNA molecule and wherein all of the different microRNA molecules target the same mRNA.

The composition may further comprise four or more, five or more, six or more, or seven or more compounds each capable of blocking the binding of a different microRNA molecule to the same target mRNA, wherein each microRNA molecule is different in structure or sequence to each other.

According to another aspect of the invention, there is provided the composition of the invention herein, for use in the prevention or treatment of asthma in a patient.

According to another aspect of the invention, there is provided a composition for use in the prevention or treatment of asthma in a patient, wherein the composition comprises at least one oligonuleotide capable of blocking a microRNA molecule selected from miR-150, miR-152 and miR-375 from binding to a target mRNA. The composition may comprise at least two oligonuleotides capable of blocking a microRNA molecule selected from miR-150, miR-152 and miR-375 from binding to a target mRNA.

According to another aspect of the invention, there is provided a method of prevention or treatment of asthma, comprising administering a composition of the invention herein.

According to another aspect of the invention, there is provided a method of prevention or treatment of asthma, comprising administering a composition comprising at least one oligonuleotide capable of blocking a microRNA molecule selected from miR-150, miR-152 and miR-375 from binding to a target mRNA. The composition may comprise at least two oligonuleotides capable of blocking a microRNA molecule selected from miR-150, miR-152 and miR-375 from binding to a target mRNA.

According to another aspect of the invention, there is provided a method of prevention or treatment of asthma, comprising administering:
a first compound capable of blocking a first microRNA molecule from binding to a target mRNA; and optionally
a second compound capable of blocking a second microRNA molecule from binding to the target mRNA,
wherein the first and second microRNA molecules are different in sequence or structure relative to each other.

The method may further comprise the administration of a third compound, wherein the third compound is capable of blocking a third microRNA molecule from binding to the same target mRNA, wherein the first and second, and third microRNA molecules are different in sequence or structure relative to each other.

The method may comprise the administration of a plurality of different compounds, wherein the plurality of compounds is capable of blocking the binding of a plurality of different microRNA molecules to the same target mRNA.

The plurality of microRNA molecules may be different in sequence or structure relative to each other.

Administration of the first compound and the second compound may be concurrently, simultaneously, or sequentially. The plurality of different compounds may be administered concurrently, simultaneously, or sequentially, or combinations thereof, relative to each other. The first compound and the second compound may be combined in a single composition. The plurality of compounds may be combined in a single composition, or combined in pairs or groups in multiple compositions.

The asthma may be viral exacerbated asthma. The patient may have, or is at risk of, a respiratory viral infection or viral exacerbated asthma. The patient may have, or is at risk of, a bacterial respiratory infection or bacterial exacerbated asthma. The patient may have, or is at risk of, allergen exacerbated asthma. The patient may have, or is at risk of, pollutant exacerbated asthma.

The administration of the composition may be before or during an asthmatic episode. The administration of the composition may be before, during or after a viral infection or viral exacerbation of the asthma. The administration may be prophylactic. The administration may be provided to a subject during a high risk period for viral exacerbation, for example in autumn and/or spring seasons; during pandemics or epidemics; during local viral outbreaks. The administration may be provided to a subject if a co-habitor, family, colleague or other group member is infected with a virus.

Prophylactic administration advantageously has no effect in the absence of viral exacerbation, yet it enhances the ability of the alveolar macrophage to detect a viral infection, and potentially prevent the infection from spreading or developing. The administration advantageously does not cause the negative secondary effects of other asthma therapies such as interferon treatment.

The composition comprising two or more compounds of two, three, four, five, six or seven compounds that bind to or block the micro RNAs may be administered simultaneously in one formulation. The combination of two three, four, five, six or seven compounds may be administered sequentially.

The composition may comprise pharmaceutically acceptable excipients. The composition may further comprise suitable excipients, carriers and/or diluents.

The composition may be delivered by intravenous or subcutaneous injection. The composition may be delivered by aerosol. An aerosol may be defined as a suspension of small particles or droplets suspended in gas or vapour.

According to another aspect of the invention, there is provided a composition comprising an antogomir or blockmir of miR-150; and/or an antogomir or blockmir of miR-152; and/or an antogomir or blockmir of miR-375.

The composition of the invention may comprise an additional active ingredient. The additional active ingredient may comprise an asthma medicament. The additional active ingredient may comprise an anti-viral medication. The additional active ingredient may comprise an antibiotic. The additional active ingredient may comprise an anti-inflammatory. The additional active ingredient may comprise a bronchodilator. The additional active ingredient may comprise a steroid. Combinations of the above actives may be provided.

The additional active ingredient may comprise a medicament selected from any of the group comprising a short-acting beta2-adrenoceptor agonist (SABA); an anticholinergic medication; an anticholinergic bronchodilator; an adrenergic agonist; a corticosteroid; a long-acting beta-adrenoceptor agonist (LABA); a leukotriene antagonist; and a mast cell stabilizer; or combinations thereof.

The additional active ingredient may comprise a medicament selected from any of the group comprising salbutamol (albuterol); terbutaline; ipratropium bromide; epinephrine; fluticasone propionate; beclometasone; salmeterol; formoterol; montelukast; zafirlukast; cromolyn sodium; cromoglycate; nedocromil; budesonide; ciclesonide; fluticasone; and mometasone; or combinations thereof.

According to another aspect of the invention, there is provided a nebuliser or inhaler comprising at least one compound capable of blocking at least one microRNA molecule selected from miR-150, miR-152 and miR-375 from binding to its mRNA target.

The nebuliser or inhaler may comprise the composition of the invention herein.

The nebuliser or inhaler may comprise an additional active ingredient. The additional active ingredient may comprise an asthma medicament. The additional active ingredient may comprise an anti-viral medication. The additional active ingredient may comprise an antibiotic. The additional active ingredient may comprise an anti-inflammatory. The additional active ingredient may comprise a bronchodilator. The additional active ingredient may comprise a steroid. Combinations of the above actives may be provided.

The additional active ingredient may comprise a medicament selected from any of the group comprising a short-acting beta2-adrenoceptor agonist (SABA); an anticholinergic medication; an anticholinergic bronchodilator; an adrenergic agonist; a corticosteroid; a long-acting beta-adrenoceptor agonist (LABA); a leukotriene antagonist; and a mast cell stabilizer; or combinations thereof.

The additional active ingredient may comprise a medicament selected from any of the group comprising salbutamol (albuterol); terbutaline; ipratropium bromide; epinephrine; fluticasone propionate; beclometasone; salmeterol; formoterol; montelukast; zafirlukast; cromolyn sodium; cromoglycate; nedocromil; budesonide; ciclesonide; fluticasone; and mometasone; or combinations thereof.

According to another aspect of the invention, there is provided a pharmaceutical composition comprising pharmaceutically acceptable excipients and a composition according to the invention herein.

According to another aspect of the invention, there is provided a composition for modifying the activity of microRNA-mediated regulation of mRNA in a cell, wherein the composition comprises:
two or more compounds capable of preventing or reducing the expression of two or more microRNA molecules which bind to the same target mRNA,
wherein the two or more microRNA molecules are different in sequence or structure relative to each other.

According to another aspect of the invention, there is provided a composition for use in the prevention or treatment of asthma in a patient, wherein the composition comprises at least one molecule capable of preventing or reducing the expression of a microRNA molecule selected from miR-150, miR-152 and miR-375 in an alveolar macrophage cell. The composition may comprise at least two molecules capable of preventing or reducing the expression of a microRNA molecule selected from miR 150, miR-152 and miR-375 in an alveolar macrophage cell.

The expression may be prevented or reduced by binding of a molecular probe, oligo or other molecule specific for the nucleic acid encoding or controlling the expression of the microRNA molecule. The expression may be prevented or reduced by mutation, removal or knock-out of the nucleic acid encoding or controlling the expression of the microRNA molecule. The mutation, removal or knock-out of the nucleic acid may be provided in the form of gene-therapy. The mutation, removal or knock-out of the nucleic acid may be provided by a nucleic acid vector delivered into the cell, for example by a modified virus.

The skilled person will understand that optional features of one embodiment or aspect of the invention may be applicable, where appropriate, to other embodiments or aspects of the invention.

There now follows by way of example only a detailed description of the present invention with reference to the accompanying drawings, in which;

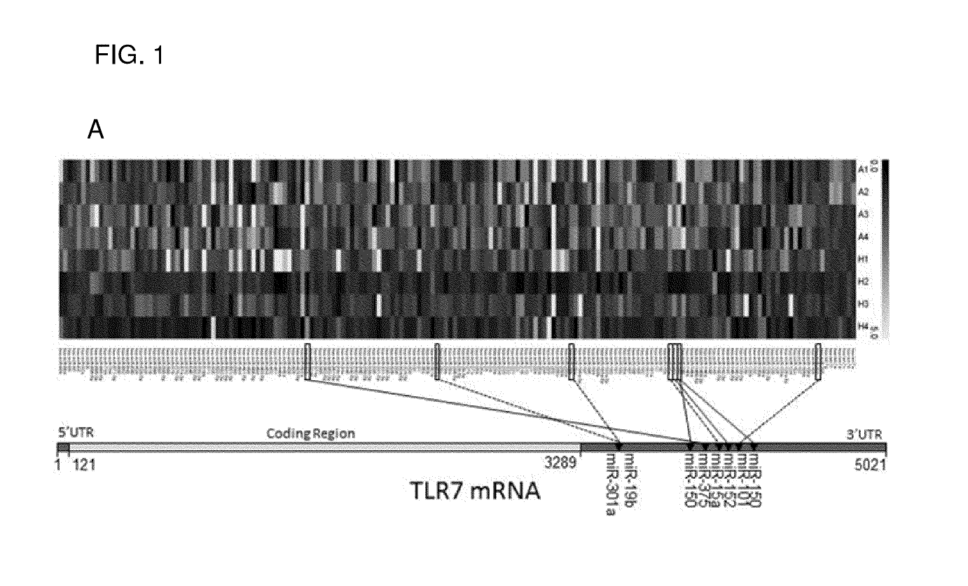

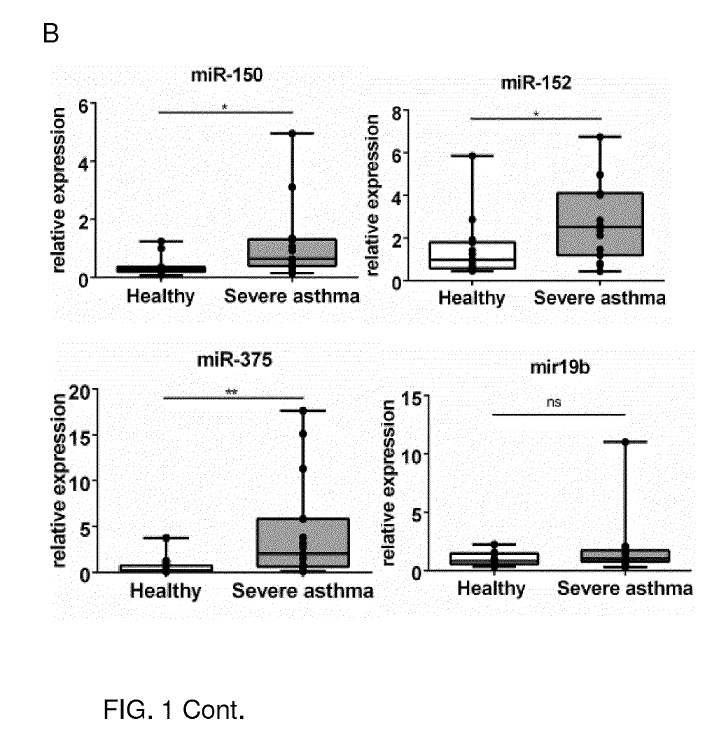

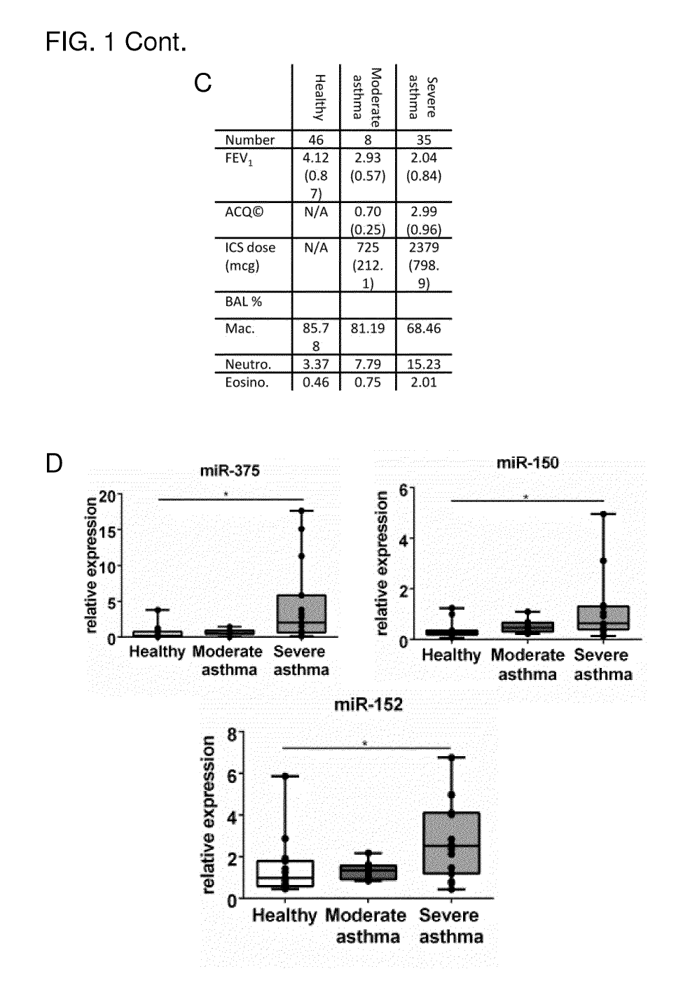

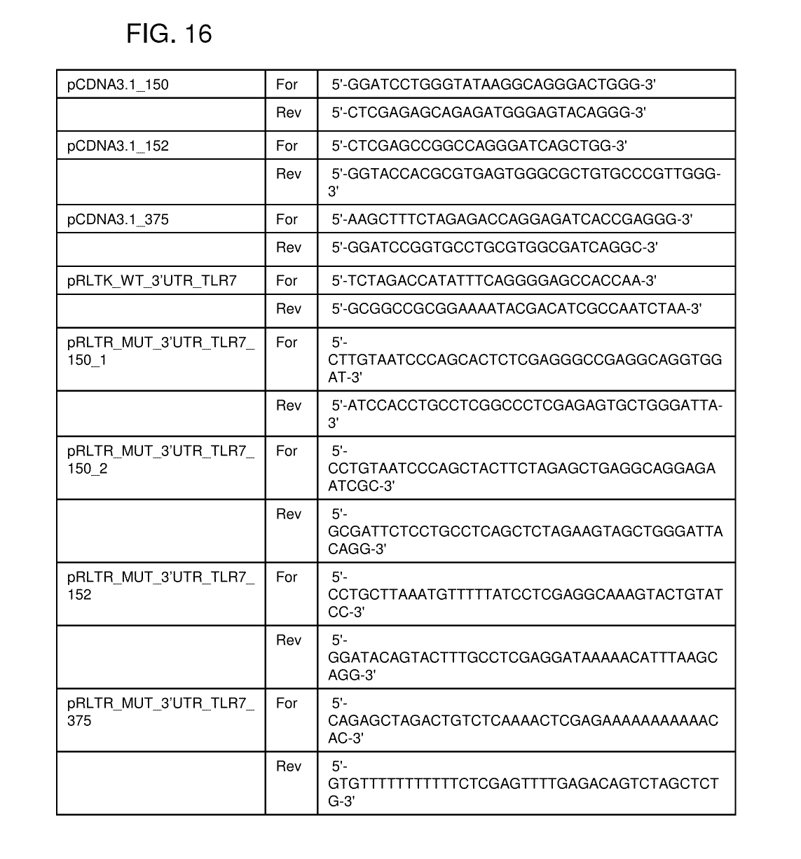

FIG. 1 shows that microRNAs are dysregulated in alveolar macrophages of severe asthmatics. (a) Heat map representing Taqman Low Density Arrays expression of microRNAs (0 to 4 fold, average=1) comparing BAL-macrophages from asthmatics (A1-4) to healthy (H1-4), showing a schematic predicted targeting of TLR7 3′UTR by microRNAs increased in asthmatics (solid line) or with a predicted score ≦−0.04 (dashed line) as in Figure S2. (b) Expression of miR-150, miR-152, miR-375 but not miR-19 is elevated in severe asthma AM (n=15) compared to healthy AM (n=15). Expression is relative to RNU44. ns=not significant, *p<0.05, **p<0.01 using two-tailed Mann Whitney test. (c) Table representing clinical data of patients participating in the study. Mac. Macrophage, Eosino. Eosinophils, Neutro. Neutrophils (SD), more details in FIG. 16. (d) Expression of miR-150, miR 152 and miR-375 in healthy (n=15), moderate asthma (n=8) and severe asthma AM (n=15). Expression is relative to RNU44. *p<0.05 using one-way ANOVA with Tukey's multiple comparison test;

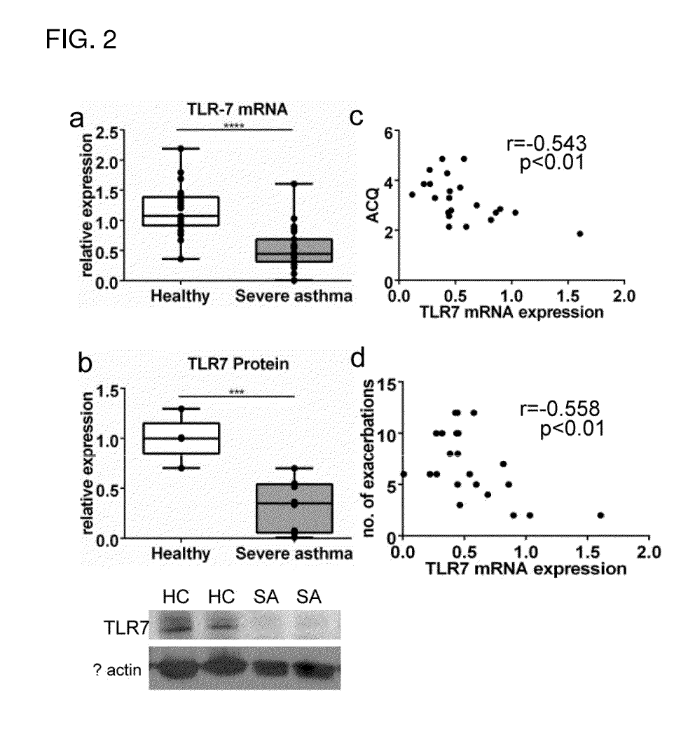

FIG. 2 shows TLR7 expression is decreased in alveolar macrophages from severe asthmatics which correlates with clinical status of patients. (a) Expression of TLR7 mRNA is reduced in severe asthma AM (n=23) compared to healthy subjects (n=23). Determined by RT-qPCR, expression is relative to GAPDH. ****p<0.0001 using two-tailed Mann Whitney test. (b) Expression of TLR7 protein is reduced in SA-AM (n=8) compared to healthy subjects (n=5). Determined by Western Blot and relative to β-actin. ***p<0.001 by two-tailed unpaired t test. Error bars, s.e.m. Panel below is representative western blot image showing the expression of TLR7 and β-actin in healthy (HC) and severe asthma (SA) AM. (c) Expression of TLR7 mRNA by SA-AM was inversely correlated with the patients ACQ. r=−0.5430, p<0.01 using two-tailed Pearson test (d) Expression of TLR7 mRNA by SA-AM was inversely correlated with the number of exacerbations experienced in the preceding 12 months. r=−0.558, p<0.01 by two-tailed Spearman test;

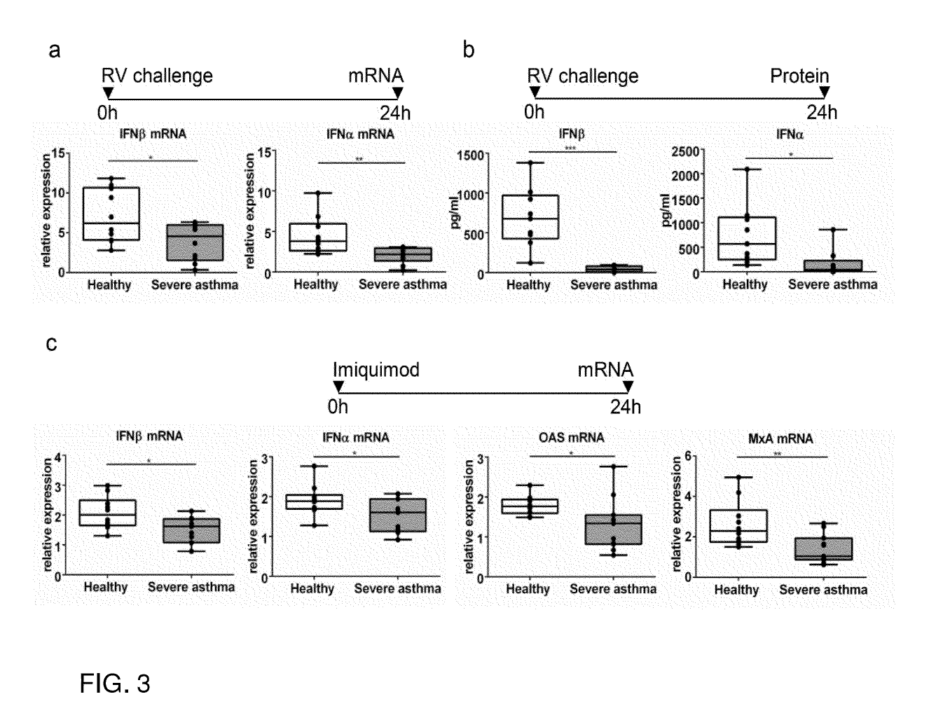

FIG. 3 Shows that alveolar Macrophages from severe asthmatics show a reduced response to viral challenge. (a) Rhinovirus-16 (RV-challenge) dependent induction of IFNβ and IFNα mRNA is reduced in Severe asthma AM (n=10) compared to healthy AM (n=10), RT-qPCR relative to GAPDH. (b) Rhinovirus-16 (RV-challenge) dependent induction of IFNβ and IFNα protein is reduced in Severe asthma AM (n=9) compared to healthy AM (n=9) (c) Imiquimod induced production of IFNβ, IFNα, OAS and M×A mRNA is reduced in Severe asthma AM (n=10) compared to healthy AM (n=11), RT-qPCR relative to GAPDH. *p<0.05, **p<0.01, ***p<0.001 by two-tailed unpaired t test;

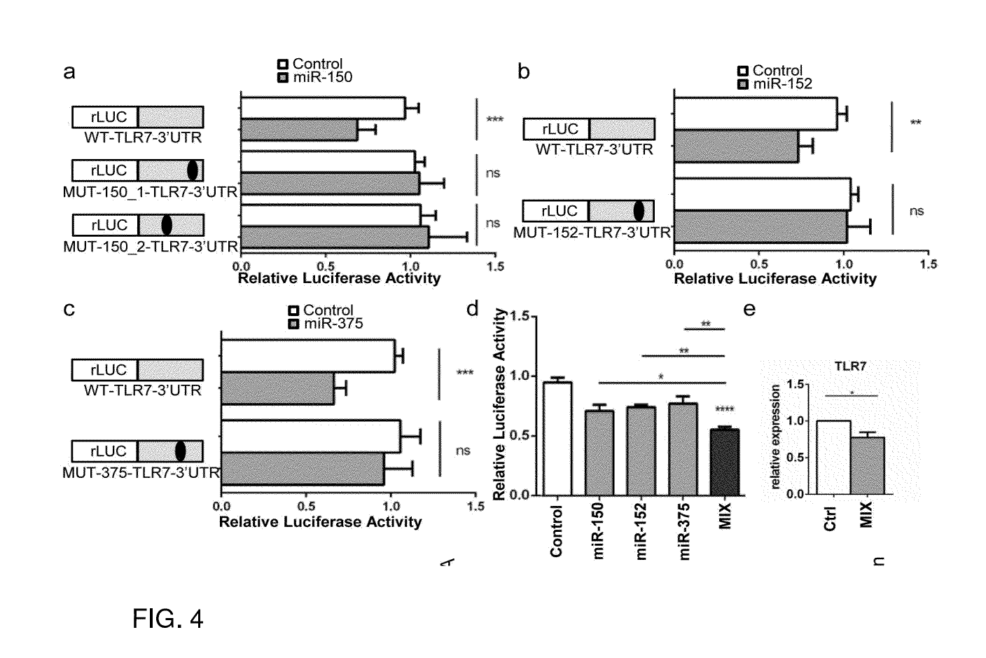

FIG. 4 Shows that miR-150, miR-152 and miR-375 directly target TLR7 and reduce its expression in alveolar macrophages. (a) Transfection of miR-150 reduces renilla-luciferase (rLUC) activity of the TLR7-reporter (WT-TLR-7 3′UTR). The effect is abrogated when the predicted binding sequence for miR-150 are mutated (MUT-150_{—}1-TLR7-3′UTR and MUT-150_{—}2-TLR7-3′UTR). (b) Transfection of miR-152 reduces rLUC activity of the TLR7-reporter (WT-TLR7-3′UTR). The effect is abrogated when the predicted binding sequence for miR-152 is mutated (MUT-152-TLR7-3′UTR) (c) Transfection of miR-375 reduces rLUC activity of the TLR7-reporter (WT-TLR7-3′UTR). The effect is abrogated when the predicted binding sequence for miR-375 is mutated (MUT-375-TLR7-3′UTR) (d) Expression of all 3 microRNAs (miR-150, miR-152 and miR-375) further reduces expression of rLUC in WT-TLR7-3′UTR, compared to the individual microRNAs. a, b, c and d (n=4); *p<0.05, **p<0.01, ***p<0.001, ****p<0.0001, ns=not significant using one-way ANOVA with Sidak's multiple comparisons test. Error bars, s.e.m. (e) TLR7 protein expression was reduced after transfection of healthy AM (n=4) with microRNA mimics for miR=150, miR-152 and miR-375 (MIX) when compared to control scrambled microRNA (Ctrl). *p<0.05 using two-tailed unpaired t test;

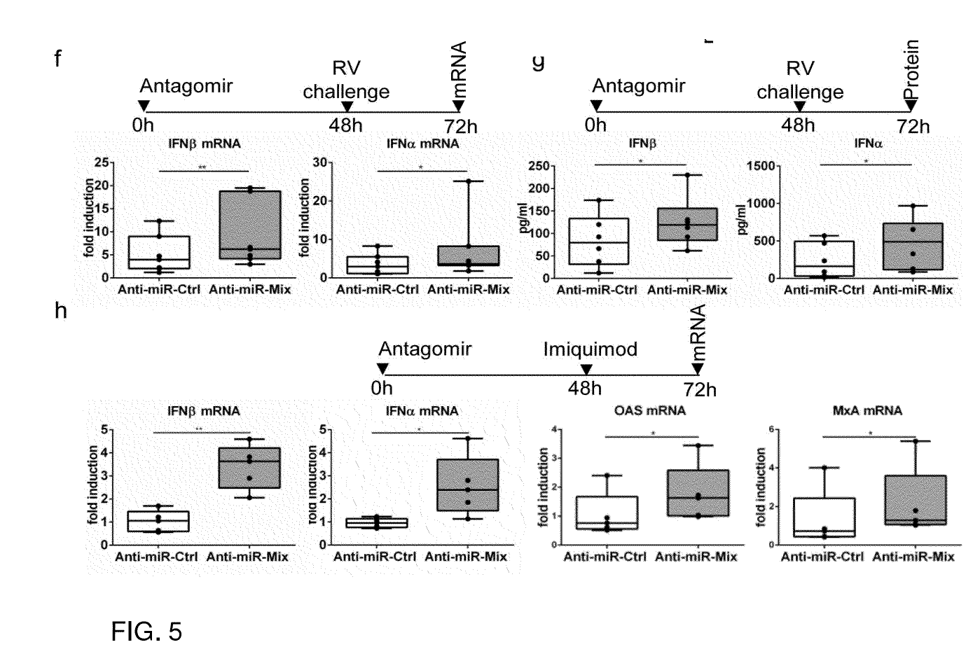

FIG. 5 Shows knockdown of miR-150, miR-152 and miR-375 rescues the TLR7-dependent response to viral infection in alveolar macrophages (a) Transfection of AM (n=7) with anti-miR-150, anti-miR-152 and anti-miR-375 increases Rhinovirus-16 (RV-challenge) induction of IFNβ and IFNα mRNA. (b) Transfection of AM (n=6) with anti-miR-150, anti-miR-152 and anti-miR-375 increases Rhinovirus-16 (RV-challenge) induction of IFNβ and IFNα protein. (c) Transfection of AM (n=5) with anti-miR-150, anti-miR-152 and anti-miR-375 increases Imiquimod induction of IFNβ IFNα, OAS and M×A mRNA.e, g, h; *p<0.05 using two-tailed unpaired t test;

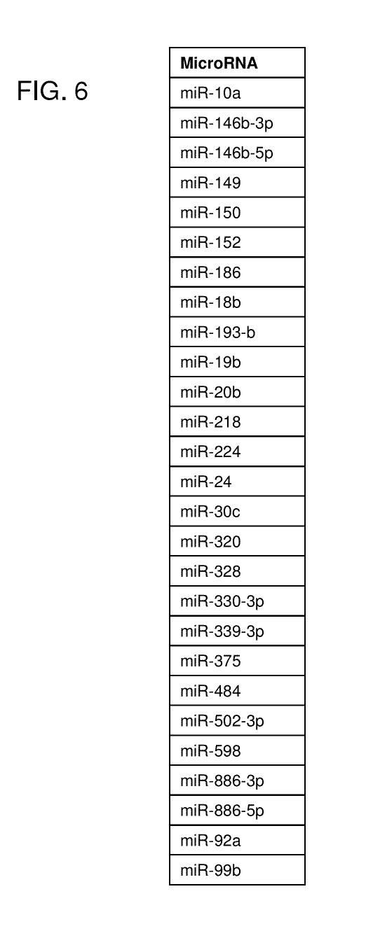

FIG. 6 Shows a list of miRNAs highlighted by the microarray as being up-regulated in asthma;

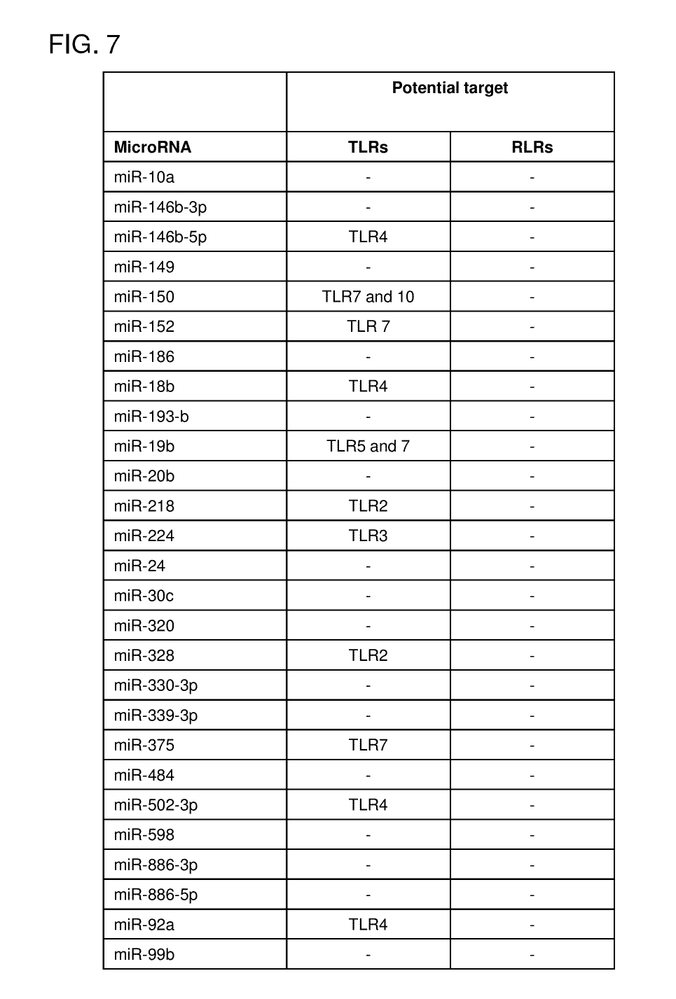

FIG. 7 Shows a putative toll-like receptor (TLR) and retinoic acid-inducible gene1-like receptors (RLRs) targets for the miRNAs highlighted in the microarray with a context score ≦−0.04 (http://www.tarcetscan.orq/vert_{—}42/);

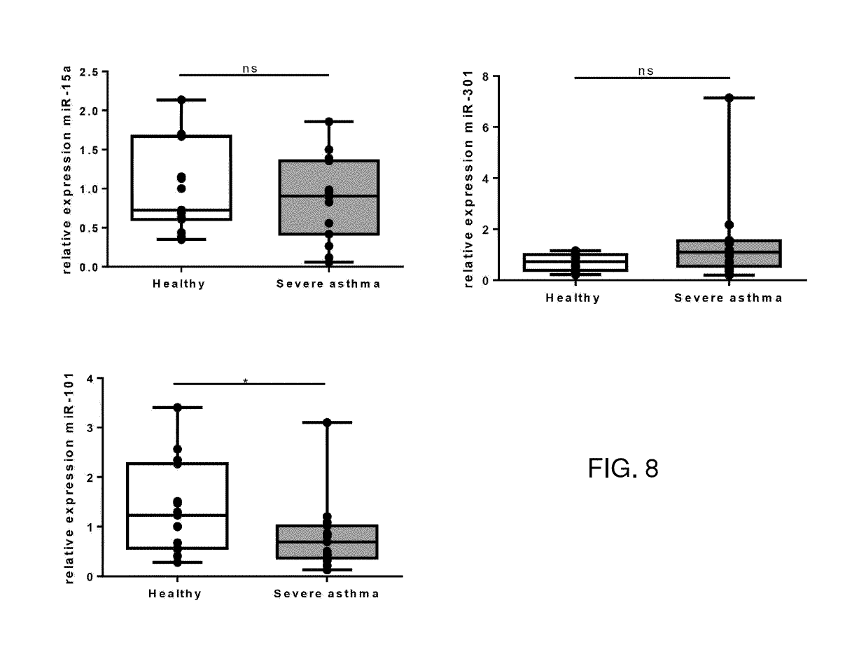

FIG. 8 Shows expression of miR-15a, miR-101 and miR-301 in healthy (n=15) and severe asthma AM (n=15). Expression is relative to RNU44. ns=not significant. *p<0.05 using two-tailed Mann Whitney. The expression of miR-144 was undetectable in both healthy (n=15) and severe asthma AM (n=15);

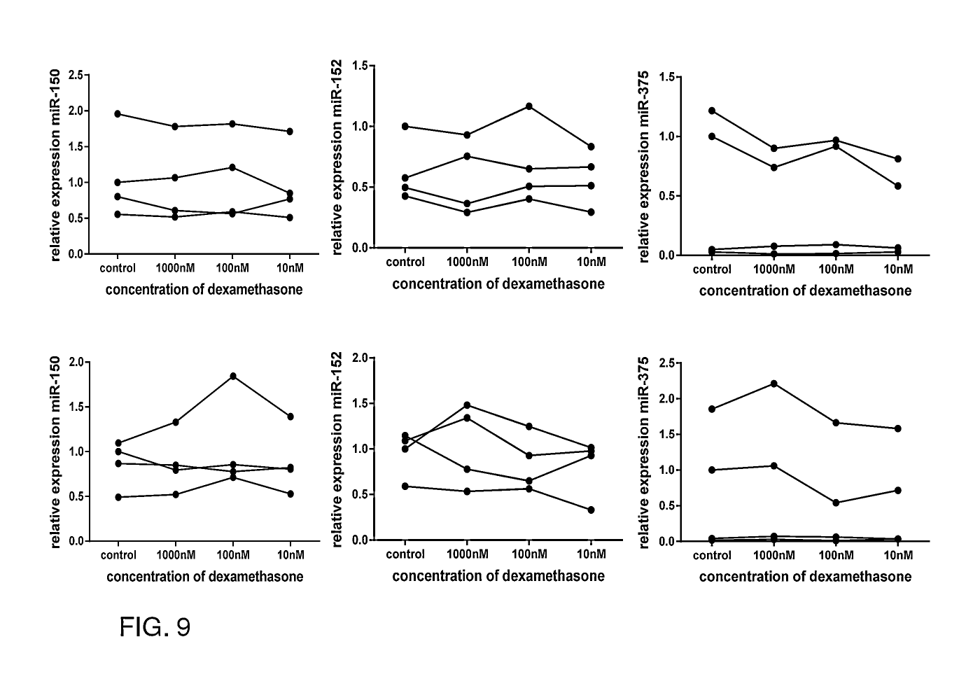

FIG. 9 Shows no change was found in the expression of miR-150, miR-152 and miR-375 in healthy AM (n=4; each line represents one donor's sample) treated with dexamethasone for 24 and 48 hours (compared to control sample), using one-way Anova with Dunnets multiple comparison;

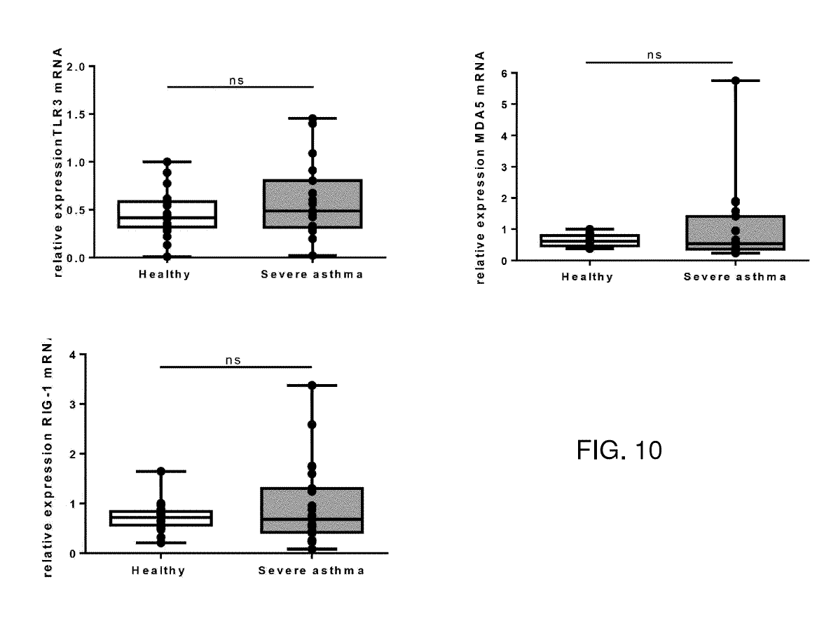

FIG. 10 Shows expression of TLR3, RIG-1 and MDA5 mRNA is similar in severe asthma (n=23) and healthy (n=23) AM. Expression is relative to GAPDH. ns=not significant using two-tailed unpaired t test;

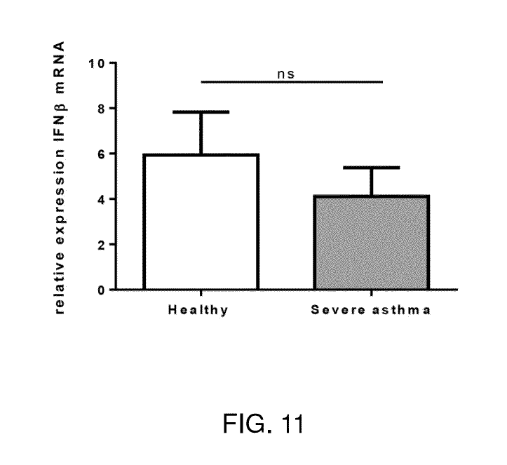

FIG. 11 Shows poly:IC induced production of IFNβ is not deficient in severe asthma AM (n=5) compared to healthy subjects (n=3). ns=not significant compared to healthy subjects by two-tailed unpaired t test. Error bars, s.e.m.;

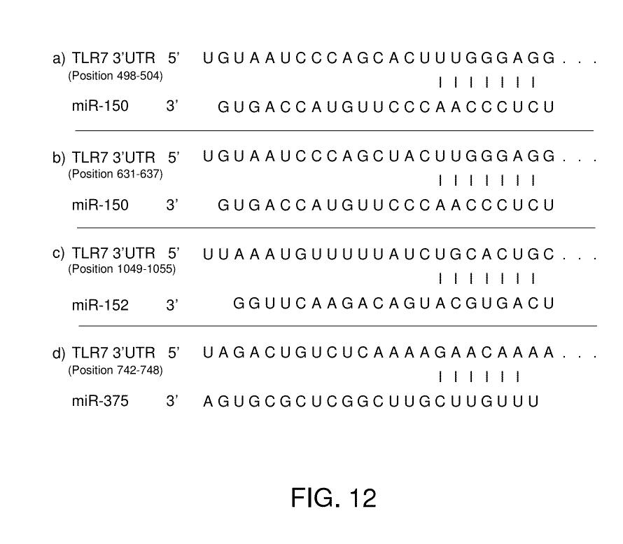

FIG. 12 Shows predicted binding sites for miR-150, miR-152 and miR-375 in the 3′UTR of TLR7 (based on Targetscan V4.2: (http://www.targetscan.org/vert_{—}42/);

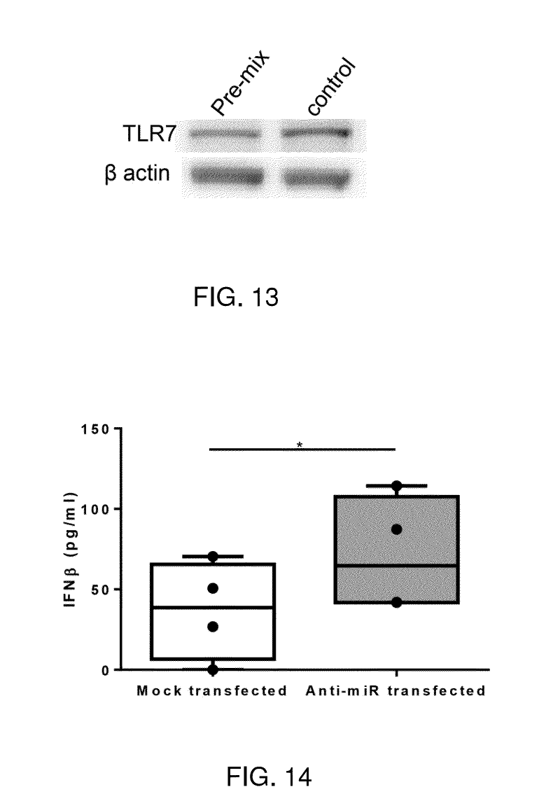

FIG. 13 Shows expression of TLR7 in healthy AM transfected with Pre-mix (pre-miR-150, pre-miR-152 and pre-miR-375) or control for 48 hours;

FIG. 14 Shows transfection of AM with anti-miR-150, anti-miR-152 and anti-miR-375 significantly increases imiquimod induced production of IFNβ in the cell supernatants compared to mock-transfected cells (n=4). *p<0.05 using two-tailed paired t test;

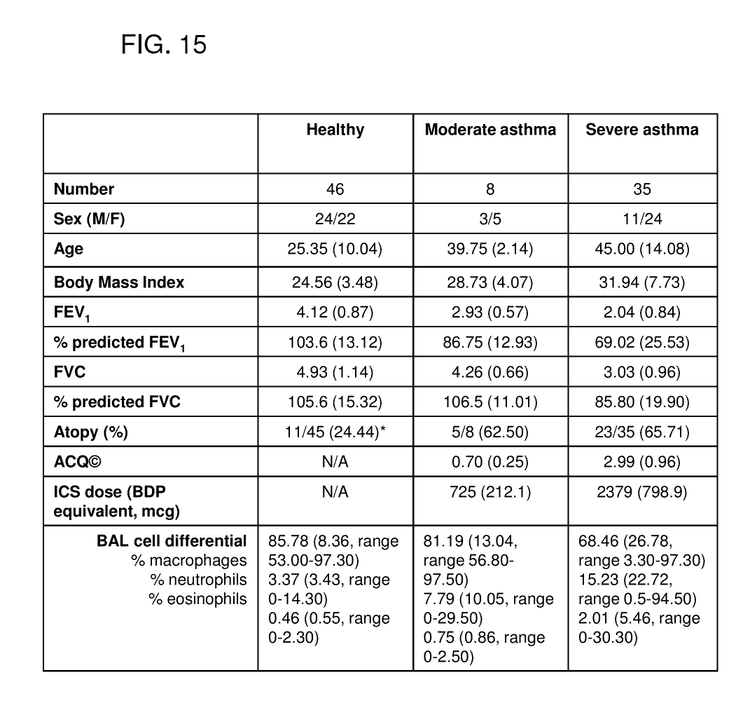

FIG. 15 Shows demographic details and clinical characteristics for subjects used. Values are means with Standard Deviation in parenthesis, except for Atopy. For Bronchoalveolar Lavage (BAL) cell differentials data range is also shown in parenthesis.*data unavailable for one subject. FEV1, forced expiratory volume in one second; FVC, forced vital capacity; ACQ, Asthma Control Questionnaire; ICS, inhaled corticosteroid; BDP, beclomethasone; BAL, bronchoalveolar lavage;

FIG. 16 Shows oligonucleotides used for cloning;

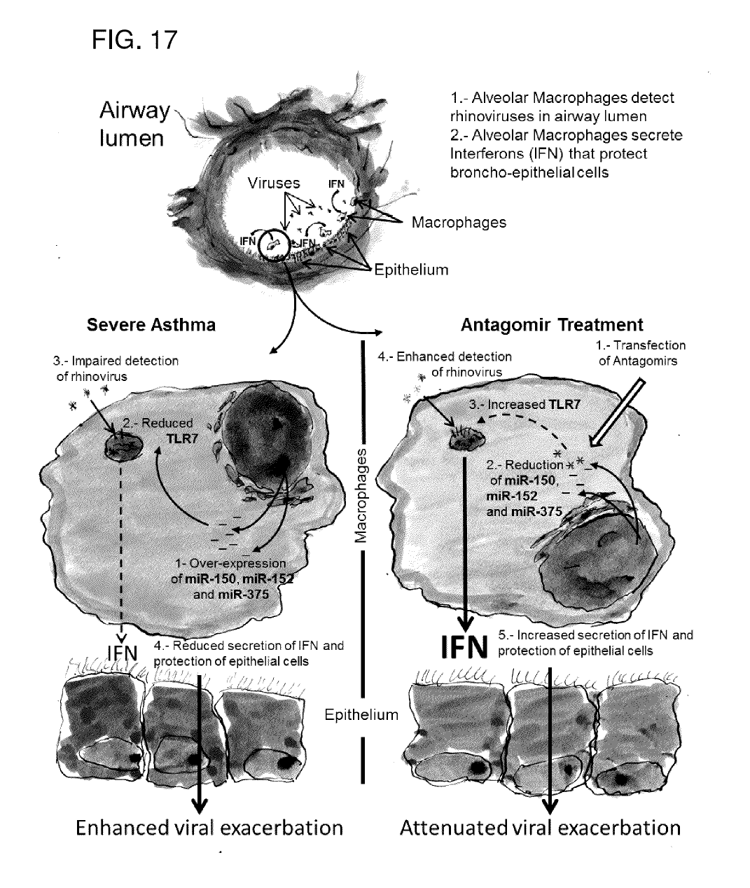

FIG. 17 Shows a schematic model proposed for the findings presented.

Sequence information

| SEQ | | |
|---|---|---|
| ID | | |
| NO | Name | Sequence |
| 1 | TLR7 3′ UTR Position | UGUAAUCCCAGCACUUUGGGAGG |
| | 498-504, 5′-3′ | |
| 2 | Mir-150 3′-5′ | GUGACCAUGUUCCCAACCCUCU |
| 3 | TLR7 3′ UTR Position | UGUAAUCCCAGCUACUUGGGAGG |
| | 631-637, 5′-3′ | |
| 4 | miR-152 3′-5′ | GGUUCAAGACAGUACGUGACU |
| 5 | TLR7 3′ UTR Position | UUAAAUGUUUUUAUCUGCACUGC |
| | 1049-1055, 5′-3′ | |
| 6 | Mir-375, 3′-5′ | AGUGCGCUCGGCUUGCUUGUUU |
| 7 | TLR7 3′ UTR Position | UAGACUGUCUCAAAAGAACAAAA |
| | 742-748, 5′-3′ | |
| 8 | pCDNA3.1_150 forward | GGATCCTGGGTATAAGGCA |
| | | GGGACTGGG |
| 9 | pCDNA3.1_150 reverse | CTCGAGAGCAGAGATGGGA |
| | | GTACAGGG |
| 10 | pCDNA3.1_152 forward | CTCGAGCCGGCCAGGGAT |
| | | CAGCTGG |
| 11 | pCDNA3.1_152 reverse | GGTACCACGCGTGAGTGGGCGC |
| | | TGTGCCCGTTGGG |
| 12 | pCDNA3.1_375 forward | AAGCTTTCTAGAGACCAGGAGA |
| | | TCACCGAGGG |
| 13 | pCDNA3.1_375 reverse | GGATCCGGTGCCTGCGTGGC |
| | | GATCAGGC |
| 14 | pRLTK_WT_3′UTR_TLR7 | TCTAGACCATATTTCAGGGGAG |
| | forward | CCACCAA |
| 15 | pRLTK_WT_3′UTR_TLR7 | GCGGCCGCGGAAAATACGACAT |
| | reverse | CGCCAATCTAA |
| 16 | pRLTR_MUT_3′UTR_TLR7_ | CTTGTAATCCCAGCACTCTCGA |
| | 150_1 forward | GGGCCGAGGCAGGTGGAT |
| 17 | pRLTR_MUT_3′UTR_TLR7_ | ATCCACCTGCCTCGGCCCTCG |
| | 150_1 reverse | AGAGTGCTGGGATTA |
| 18 | pRLTR_MUT_3′UTR_TLR7_ | CCTGTAATCCCAGCTACTTCTAG |
| | 150_2 forward | AGCTGAGGCAGGAGAATCGC |
| 19 | pRLTR_MUT_3′UTR_TLR7_ | GCGATTCTCCTGCCTCAGCTCT |
| | 150_2 reverse | AGAAGTAGCTGGGATTACAGG |
| 20 | pRLTR_MUT_3′UTR_TLR7_ | CCTGCTTAAATGTTTTTATCCT |
| | 152 forward | CGAGGCAAAGTACTGTATCC |
| 21 | pRLTR_MUT_3′UTR_TLR7_ | GGATACAGTACTTTGCCTCGAG |
| | 152 reverse | GATAAAAACATTTAAGCAGG |
| 22 | pRLTR_MUT_3′UTR_TLR7_ | CAGAGCTAGACTGTCTCAAAA |
| | 375 forward | CTCGAGAAAAAAAAAAACAC |
| 23 | pRLTR_MUT_3′UTR TLR7_ | GTGTTTTTTTTTTTCTCGAGT |
| | 375 reverse | TTTGAGACAGTCTAGCTCTG |

### miR SEQUENCE INFORMATION

Sequences and accession numbers as provided on miRBase.org.

>hsa-mir-150 (Accession No. MI0000479)
SEQ ID NO: 24 (miR-150)
CUCCCCAUGGCCCUGUCUCCCAACCCUUGUACCAGUGCUGGGCUCAG
ACCCUGGUACAGGCCUGGGGGACAGGGACCUGGGGAC
>hsa-mir-152 (Accession No. MI0000462)
SEQ ID NO: 25 (miR-152)
UGUCCCCCCCGGCCCAGGUUCUGUGAUACACUCCGACUCGGGCUCUG
GAGCAGUCAGUGCAUGACAGAACUUGGGCCCGGAAGGACC
>hsa-mir-375 (Accession No. MI0000783)
SEQ ID NO: 26 (miR-375)
CCCCGCGACGAGCCCCUCGCACAAACCGGACCUGAGCGUUUUGUUCG
UUCGGCUCGCGUGAGGC

### Introduction

Asthma exacerbations are predominantly triggered by viral infections, reflecting defective airway innate immunity. Reduced virus-induced production of interferons by airway cells appears to be a critical factor, but the underlying mechanisms are not known. A disregulation in the expression of several microRNAs has been observed in asthma. The implications of this dysregulation were investigated focusing on different microRNAs in different cells types and processes. This approach has facilitated the discovery of synergies or collaborations between multiple microRNAs. It has been found that multiple microRNAs may be used simultaneously as therapeutic targets, eliciting a more controlled response that individual microRNAs. For example, the expression of TLR7 in alveolar macrophages is shown to be reduced because a group of three co-operating microRNAs (miR-150, miR-152 and miR-375) are over-expressed. Targeting these miRNAs restores TLR7 expression and corrects the impaired innate response.

### Results and Discussion

MicroRNAs Predicted to Target TLR7 are Up-Regulated in Macrophages from Asthmatics.

Alveolar macrophages (AM) were purified from bronchoalveolar lavage (BAL) miRNA microarray analyses were performed on samples from both healthy and asthmatic volunteers. This identified 27 miRNAs that were differentially up-regulated in AM from asthmatics (FIG. 1a and FIG. 6). Using in silico analysis (Lewis et al., 2005, Cell 120:15-20) the putative targets for these miRNAs were examined. The search was concentrated on toll-like receptors (TLRs) and retinoic acid-inducible gene 1-like receptors (RLRs), as alterations in their expression could reduce resistance to infection by viruses or bacteria resulting in disease exacerbations (FIG. 7). This search revealed that four of the up-regulated miRNAs targeted TLR7, which is an endosomal TLR that is activated by single-stranded RNA, for example from rhinoviruses (RV). This pattern-recognition receptor (PRR) was studied further and it was hypothesised that increased expression of some/all four of these miRNAs could lead to a significant reduction in the expression of TLR7 in AM from individuals with severe asthma (SA-AM), thereby impairing the ability of these cells to sense the presence of rhinovirus with a resulting reduction in production of IFN.

Quantitative PCR (qPCR) on these four miRNAs confirmed that the expression of three, namely miR-150, miR-152 and miR-375, was significantly increased in severe asthmatic-AM, while the expression of the fourth, miR19b, was similar in severe asthmatic-AM and healthy AMs (FIG. 1b). Subsequently, the expression of other miRNAs (miR-15a, miR-101, miR-301 and miR-144) was evaluated, where they were predicted by bioinformatics analysis to be of relevance to TLR7, but which had not shown differential expression in the original array, and found no significant increase in severe asthmatic-AM (FIG. 8). Attention was focused on miRs-150, -152 and -375. The severe asthmatics were all on treatment with high doses of inhaled corticosteroids (ICS). Therefore, it was initially addressed whether ICS use provided an explanation for these findings in uncontrolled glucocorticosteroid treated asthma by also assessing expression of miRs-150, -152 and -375 in AM from asthmatics taking ICS who had well controlled disease, as assessed by the asthma control questionnaire© (ACQ), and who had no history of frequent disease exacerbations (FIG. 1c). In contrast to the findings in uncontrolled asthma, this identified that the expression of these three index miRNAs was not different in AM between patients with controlled asthma and healthy subjects, indicating that glucocorticoid therapy cannot explain the observed finding in severe asthma (FIG. 1d). The ex vivo dexamethasone treatment was also shown to not alter the expression of miRs-150, -152 and -375 in healthy AMs (FIG. 9).

TLR7 Expression is Reduced in Asthmatic Macrophages and Co-Relates with Clinical Status.

According to the in silico prediction above, the increased expression of miRs-150, -152 and -375 would result in reduced levels of TLR7, impairing antiviral response to rhinovirus in severe asthmatics. Therefore, the expression of TLR7 in SA-AM was evaluated and a reduction at both the mRNA and protein level was found as compared to that in healthy AM (FIG. 2a,b). Consistent with the clinical relevance of this finding, TLR7 mRNA expression in SA-AM correlated inversely with both the ACQ score of patients (FIG. 2c) and the number of exacerbations the patient had experienced in the previous year (FIG. 2d). Signalling by TLRs and TLR7 has been linked to asthma previously supporting the importance of the results. For the first time, the TLR7 expression is shown to be reduced, that this takes place in the physiologically relevant cell, the AM, and that it is correlated to clinical data. This places TLR7 and the AM as key in asthma exacerbation pathology and likely to be responsible for the asthmatic anti-viral innate immune defect.

TLR7-Dependent Induction of IFN and IFNα is Impaired in Macrophages from Asthmatics.

It was next investigated whether there was a functional impairment in the response to virus exposure of macrophages from severe asthmatics. The AM was challenged with rhinovirus (RV-16) in vitro and it was found that RV-induction of IFNα and IFNβ mRNA as well as secreted protein was significantly reduced from SA-AMs compared to healthy AMs (FIG. 3a,b). IFN production is also triggered by PRRs other than TLR7, including TLR3, retinoic acid-inducible gene I (RIG-I) and melanoma differentiation associated gene 5 (MDA5), all of which are activated by viral RNA. The expression of these 3 PRRs was evaluated and it was found that there was no difference at the mRNA level, in the expression of TLR3, RIG-1 and MDA5 in AM from SA and healthy non-asthmatic volunteers (FIG. 10). Furthermore, no differences were observed between these groups in IFNβ induction by Poly:IC; a synthetic dsRNA that activates TLR3, RIG-1 and MDA-5 (FIG. 11). Thus, the impaired IFN response to virus challenge by SA-AMs appear to relate to the reduced expression of TLR7. To confirm this, AMs from healthy and SA subjects were treated in vitro with imiquimod, a synthetic TLR7 agonist (Lee et al., 2003, Proceedings of the National Academy of Sciences of the United States of America 100:6646-6651) and the production of IFNα and IFNβ was assessed. It was found that imiquimod-induced production of IFNα and IFNβ mRNA was significantly reduced in SA-AMs compared to healthy AM (FIG. 3c). Type I IFNs also induce the production of 2′5′-oligoadenylate synthetase (OAS) and myxovirus resistance protein A (M×A), important effectors of the IFN response (Der et al., 1998; Platanias, 2005). The results also show that imiquimod-induced expression of OAS and M×A was significantly reduced in SA-AMs compared to AMs from healthy subjects (FIG. 3c). Taken together, these data show the role played by AM in the deficient response to virus in severe asthmatics and are indicative of the central role played by TLR7.

### MiRs-150, -152 and -375 Directly Target the 3′UTR and Reduce Protein Expression of TLR7.

Once it was confirmed that the in silico prediction that over-expression of miRs-150, -152 and -375 in SA-AMs (FIG. 1b) would result in deficient expression of TLR7 in these cells, and that this was both clinically and functionally relevant (FIG. 2), the next aim was to demonstrate a direct molecular link between the over-expression of these miRNAs and the deficiency in TLR7. Bioinformatic tools predicted that miR-150 targets the 3′ UnTranslated region (3′-UTR) of TLR7 at two different sites while miR-152 and miR-375 appear to target one site each (FIG. 12). Luciferase reporter constructs were generated with the 3′UTR sequence of TLR7 and it was found that the over-expression of miRs-150, -152 and -375 led to a significant decrease in luciferase activity (FIG. 4a-c). Furthermore, this response was abrogated when the binding sites for the 3 miRNAs were mutated. Next, it was shown that when all three miRNAs were overexpressed together, there was a significantly greater decrease in luciferase activity, suggesting that they are co-operating to reduce the expression of TLR7 (FIG. 4d). Finally, it was shown that over-expression of the three miRNAs in healthy primary AM leads to a substantial reduction in the expression of TLR7 at the protein level (FIG. 4e and FIG. 13). This provides direct evidence that these 3 miRNAs do indeed target the 3′UTR of TLR7, reducing the expression of this receptor.

Antagomirs Against miRs-150, -152 and -375 Restore IFN Response to Virus in Alveolar Macrophages.

These findings highlight the need for novel therapeutic approaches to restoring the normal innate immune response in order to prevent virally-induced disease exacerbation in asthma. On the basis of these findings, the knock-down of miRs-150, -152 and -375, would restore TLR7 expression and therefore function, thereby ameliorating the defective IFN production by AM in response to viral challenge. Therefore, specifically to test this, AMs were transfected with three antagomirs, anti-miR-150, anti-miR-152 and anti-miR-375. When the transfected cells were then challenged with rhinovirus they showed significantly augmented production of IFNα and IFNβ mRNA and secreted protein compared to mock transfected cells (FIG. 5a,b).

Furthermore, when these transfected AM were challenged with imiquimod, a similar, significant increase in the mRNA production of IFNα, IFNβ, M×A and OAS was seen (FIG. 5c) as was IFNβ protein (FIG. 14) in cell supernatants. These findings thus indicate the potential for inhaled antagomir therapy in severe asthma as a novel approach to correct the impaired innate anti-viral response of AMs and thereby prevent disease exacerbation.

For the first time, the specific molecular role of microRNAs in human asthma has been demonstrated. This work not only delivers a cellular and molecular understanding of the role of miRs-150, -152 and -375 in human asthma but provides real therapeutic potential for treatment of severe asthmatics with specific antagomirs to restore their anti-viral innate immunity. Furthermore, the targeting of the three microRNAs studied, which act cooperatively, is likely to limit any off target effects.

In conclusion, bioinformatic predictions and molecular tools with a broad applicability were employed to identify a novel mechanism involved in the deficient innate immune response to virus in asthma. A response that is driven by miRNA-mediated deficiency in the expression of an important PRR in macrophages, namely TLR7. More importantly, it has been shown that by manipulating the expression of miRNAs in AMs, the defective IFN response to virus can be ameliorated, making it an extremely promising and novel treatment for the future prevention of disease exacerbation in asthma.

### Materials and Methods

Study subjects—adults with severe asthma, moderate asthma and healthy subjects were recruited. Severe asthma subjects were recruited from the MRC funded Wessex Severe Asthma Cohort and Difficult Airways clinic held at University Hospitals Southampton Foundation Trust, while subjects in the latter 2 groups were recruited through local advertising. The study was approved by the Southampton and South West Hampshire Local Research Ethics Committee and all subjects gave written informed consent. Baseline spirometry was measured using the Jaeger Masterscreen with Viasys® software. Healthy and moderate asthma subjects underwent methacholine (Stockport Pharmaceuticals) challenge testing to assess airway responsiveness. Skin prick testing was performed using allergen extract from the following: aspergillus fumigatus, alternaria tenius, birch tree pollen, mixed grasses, mixed tree pollen, rape pollen, weed pollen, dermatophagoides pteronyssinus, dermatophagoides farinae, dog fur, cat dander and horse fur, along with positive (histamine) and negative (saline) control (all from Allegopharma, Germany).

Alveolar Macrophages Subjects underwent flexible bronchoscopy in accordance with established guidelines (British Thoracic Society Bronchoscopy Guidelines Committee, 2001). Bronchoalveolar lavage (BAL) was performed by instilling 120 mls (6×20 ml aliquots) pre-warmed (37° C.) normal saline into the right upper lobe. Cells from BAL were washed immediately and resuspended in RPMI 1640 with Glutamax (Invitrogen) containing 10% FCS and antibiotics (penicillin and streptomycin-P/S) at a concentration of 1×106/ml and placed at 37° C. in a humidified 5% CO2 incubator for 2 hours to allow for the AM to adhere. AM were collected in TRI-Reagent (for RNA isolation, Applied Biosystems) and NP-40 protein lysis buffer (Invitrogen, supplemented with PMSF, Sigma-Aldrich, and protease inhibitor cocktail, Sigma-Aldrich).

For the functional studies, AM were cultured in RPMI 1640 with Glutamax containing 10% FCS and antibiotics (P/S). They were then incubated with Imiquimod (5 μg/ml, Invivogen) or rhinovirus-16 at an MOI of 0.6 (or UV-inactivated rhinovirus-16) or polyl:C (10 μg/ml, Invivogen). After 24 hours supernatants were harvested for future evaluation of IFNα and IFNβ protein concentration. Adherent cells (AM) were collected in TRI-Reagent.

For the transfection studies, AM were cultured in RPMI 1640 with Glutamax containing 10% FCS and antibiotics (P/S). They were transfected (transfection occurs by natural phagocytosis or passive diffusion, no transfection reagent used) with a combination of anti-miR-150, anti-miR-152 and anti-miR-375 (50 nM each) or scrambled control (150 nM, Life Technologies). At 48 hours cells were treated with imiquimod, rhinovirus-16 or poly:IC at the concentrations stated above for 24 hours. At this point adherent cells were collected in TRI-Reagent and supernatants harvested.

For transfection studies to evaluate changes TLR7 protein expression, healthy AM were transfected with pre-miR-150, pre-miR-152 and pre-miR-375 (500 nM each, Life Technologies) or scrambled control. At 48 hours macrophages were lysed in NP-40 protein lysis buffer (supplemented with PMSF and protease inhibitor cocktail).

All samples were stored at −80° C. until use.

To study the effects of steroids on miRNAs AM from healthy subjects were treated with dexamethasone (Sigma Aldrich, 1000 nM, 100 nM, 10 nM) and incubated at 37° C. with 5% CO2. Cells were collected at 24 and 48 hours in TRI-Reagent and stored at −80° C. until analysis.

RNA Extraction and qPCR

Total RNA was isolated using TRI-Reagent. For miRNA analysis 5 ng of RNA was reverse transcribed using the High Capacity cDNA Reverse Transcription Kit with specific stem loop primers for each miRNA. All miRNA data was normalized to the internal control RNU44. For mRNA analysis 200 ng of RNA was reverse transcribed using the High Capacity cDNA Reverse Transcription Kit. GAPDH was used as an internal control. qPCR was performed using Taqman® Universal PCR Mastermix on a 7900HT Fast Real-time PCR system (Applied Biosystems) and changes in the expression of miRNA and mRNA were expressed as fold change relative to control. All reagents from Life Technologies.

### MiRNA Array

The Life Technologies Taqman® Low Density Array system was used following manufacturer's instructions. Briefly, 500 ng of total cellular RNA was was reversed transcribed using Megaplex™ RT Primers and the cDNA was then loaded onto the array card for PCR amplification. Arrays were carried out 4 healthy and 4 asthmatic subjects. Results were displayed as a Heat map using MultiExperiment Viewer (MeV: http://www.tm4.org/mev/).

### Bioinformatic Analysis

MiRNA targets were predicted using Targetscan 4.2 (http://www.targetscan.org/vert_{—}42/) with default options.

### Protein Expression Analysis

Western blotting and densitometric analysis was performed as previously described (Martinez-Nunez et al., 2009) the following antibodies were used: anti-TLR7 (1:500, Abcam), anti-mouselgG-HRP (Fisher Scientific) and anti-β-actin-peroxidase (1:25,000, Sigma-Aldrich).

The Verikine™ IFNβ enzyme-linked immunoabsorbant assay (range 25-2000 pg/ml) was used which employs a sandwich immunoassay. It was done using manufacturer's instructions and standards, samples and blanks were run in duplicate.

Meso Scale Discovery singleplex kits were also used for evaluation of IFNα and IFNβ concentrations in cell supernatants. It was done using manufacturer's instructions and standards, samples and blanks were run in duplicate.

### Generation of Vectors and Luciferase Assays

The 3′UTR of TLR7, containing the predicted miR-150, miR-152 and miR-375 seed sequences was amplified from human genomic DNA and cloned into XbaI and NotI sites of the pRLTK vector (Promega) and called pRLTK_WT_{—}3′UTR_TLR7. Site-directed mutagenesis was performed on this vector to create vectors containing mutations in the seed sequence(s) for miR-150 (pRLTK_MUT_{—}3′UTR_TLR7_{—}150_{—}1, pRLTK_MUT_{—}3′UTR_TLR7_{—}150_{—}2), miR-152 (pRLTK_MUT_{—}3′UTR_TLR7_{—}152) and mir375 (pRLTK_MUT_{—}3′UTR_TLR7_{—}375). For pCDNA3.1_{—}150, pCDNA3.1_{—}152 and pCDNA3.1_{—}375 genomic DNA was amplified by PCR and DNA fragments cut with different pairs of enzymes were cloned into BamHI/XhoI, XhoI/KpnI and XbaI/BamHI pCDNA3.1 multi-cloning sites respectively. In order to generate pCDNA3.1_MIX, these fragments were cloned simultaneously in pCDNA3.1(−) using XbaI/KpnI (order of cloning from promoter: miR-275, miR-150 and miR152).

Primers used to generate the vectors in FIG. 16:

Transfections were performed 3 times in duplicate. HeLa cells were co-transfected with the luciferase reporter constructs containing either the WT (wild-type) or MUT (3′UTR mutants) and 800 ng of pCDNA3.1_{—}150 or pCDNA3.1_{—}152 or pCDNA3.1_{—}375. Transfections were carried out using Superfect (Qiagen) and normalization was performed using pGL3 (25 ng/well, Promega). Transfected cells were cultured for 24 hours after which measurements were made using the Dual-Luciferase® Reporter Assay System (Promega).

1. A composition for modifying the activity of microRNA-mediated regulation of mRNA in a cell, wherein the composition comprises:
two or more compounds capable of blocking two or more microRNA molecules from binding to the same target mRNA,
wherein the two or more microRNA molecules are different in sequence or structure relative to each other.

2. The composition according to claim 1, wherein the cell is an alveolar macrophage.

3. The composition according to claim 1, wherein the cell is a lung epithelial cell.

4. The composition according to claim 1, wherein at least one compound is arranged to bind to a microRNA molecule to prevent it from engaging with the target mRNA.

5. (canceled)

6. The composition according to claim 1, wherein at least one compound is arranged to bind to the target mRNA in order to block a binding site of a microRNA molecule.

7. (canceled)

8. The composition according to claim 1, wherein at least one microRNA molecule is selected from the group consisting of miR-150, miR-152 and miR-375.

9. The composition according to claim 1, wherein the two or more microRNA molecules comprise two or more microRNA molecules selected from the group consisting of miR-150, miR-152 and miR-375.

10. The composition according to claim 1, wherein the two or more microRNA molecules comprise three or more microRNA molecules selected from the group consisting of miR-150, miR-152 and miR-375.

11-14. (canceled)

15. The composition according to claim 1, wherein the mRNA encodes TLR7 or RipK1.

16. The composition according to claim 1, wherein at least one compound is an oligonucleotide, antagomir or blockmir.

17. The composition according to claim 1, wherein the two or more compounds are selected from the group consisting of oligonucleotides, antagomirs and blockmirs, or combinations thereof.

18. The composition according to claim 1, wherein at least one compound is an oligonucleotide comprising a sequence complementary to a sequence selected from the group consisting of SEQ ID NO. 24, SEQ ID NO. 25, and SEQ ID NO. 26.

19. The composition according to claim 1, wherein the two or more compounds comprise two or more oligonucleotides comprising a sequence complementary to a sequence selected from the group consisting of SEQ ID NO. 24, SEQ ID NO. 25, and SEQ ID NO. 26.

20. (canceled)

21. The composition according to claim 18, wherein the sequence is complementary across at least about 5 nucleic acid residues.

22. The composition according to claim 1, wherein at least one compound is a small molecule or a peptide.

23. (canceled)

24. The composition according to claim 1, further comprising an additional active ingredient; and optionally wherein the additional active ingredient comprises an asthma medicament; and further optionally wherein the additional active ingredient is a steroid.

25-30. (canceled)

31. A method of prevention or treatment of asthma, comprising administering:
a first compound capable of blocking a first microRNA molecule from binding to a target mRNA; and optionally
a second compound capable of blocking a second microRNA molecule from binding to the target mRNA,
wherein the first and second microRNA molecules are different in sequence or structure relative to each other.

32. The method according to claim 31, further comprise the administration of a third compound, wherein the third compound is capable of blocking a third microRNA molecule from binding to the same target mRNA, and wherein the first and second, and third microRNA molecules are different in sequence or structure relative to each other.

33. The method according to claim 31, the method comprising the administration of a plurality of different compounds, wherein the plurality of compounds is capable of blocking the binding of a plurality of different microRNA molecules to the same target mRNA.

34. (canceled)

35. An inhaler or nebuliser comprising at least one compound capable of blocking at least one microRNA molecule selected from miR-150, miR-152 and miR-375 from binding to its mRNA target.

36-38. (canceled)


SEQUENCE LISTING (26 sequences)

SEQ ID NO: 1 - RNA, 23 nt, Homo sapiens
uguaaucccagcacuuugggagg

SEQ ID NO: 2 - RNA, 22 nt, Homo sapiens
gugaccauguucccaacccucu

SEQ ID NO: 3 - RNA, 23 nt, Homo sapiens
uguaaucccagcuacuugggagg

SEQ ID NO: 4 - RNA, 21 nt, Homo sapiens
gguucaagacaguacgugacu

SEQ ID NO: 5 - RNA, 23 nt, Homo sapiens
uuaaauguuuuuaucugcacugc

SEQ ID NO: 6 - RNA, 22 nt, Homo sapiens
agugcgcucggcuugcuuguuu

SEQ ID NO: 7 - RNA, 23 nt, Homo sapiens
uagacugucucaaaagaacaaaa

SEQ ID NO: 8 - DNA, 28 bp, Artificial Sequence
  Primer
ggatcctgggtataaggcagggactggg

SEQ ID NO: 9 - DNA, 27 bp, Artificial Sequence
  Primer
ctcgagagcagagatgggagtacaggg

SEQ ID NO: 10 - DNA, 25 bp, Artificial Sequence
  Primer
ctcgagccggccagggatcagctgg

SEQ ID NO: 11 - DNA, 35 bp, Artificial Sequence
  Primer
ggtaccacgcgtgagtgggcgctgtgcccgttggg

SEQ ID NO: 12 - DNA, 32 bp, Artificial Sequence
  Primer
aagctttctagagaccaggagatcaccgaggg

SEQ ID NO: 13 - DNA, 28 bp, Artificial Sequence
  Primer
ggatccggtgcctgcgtggcgatcaggc

SEQ ID NO: 14 - DNA, 29 bp, Artificial Sequence
  Primer
tctagaccatatttcaggggagccaccaa

SEQ ID NO: 15 - DNA, 33 bp, Artificial Sequence
  Primer
gcggccgcggaaaatacgacatcgccaatctaa

SEQ ID NO: 16 - DNA, 40 bp, Artificial Sequence
  Primer
cttgtaatcccagcactctcgagggccgaggcaggtggat

SEQ ID NO: 17 - DNA, 36 bp, Artificial Sequence
  Primer
atccacctgcctcggccctcgagagtgctgggatta

SEQ ID NO: 18 - DNA, 43 bp, Artificial Sequence
  Primer
cctgtaatcccagctacttctagagctgaggcaggagaatcgc

SEQ ID NO: 19 - DNA, 43 bp, Artificial Sequence
  Primer
gcgattctcctgcctcagctctagaagtagctgggattacagg

SEQ ID NO: 20 - DNA, 42 bp, Artificial Sequence
  Primer
cctgcttaaatgtttttatcctcgaggcaaagtactgtatcc

SEQ ID NO: 21 - DNA, 42 bp, Artificial Sequence
  Primer
ggatacagtactttgcctcgaggataaaaacatttaagcagg

SEQ ID NO: 22 - DNA, 41 bp, Artificial Sequence
  Primer
cagagctagactgtctcaaaactcgagaaaaaaaaaaacac

SEQ ID NO: 23 - DNA, 41 bp, Artificial Sequence
  Primer
gtgtttttttttttctcgagttttgagacagtctagctctg

SEQ ID NO: 24 - RNA, 84 nt, Homo sapiens
cuccccauggcccugucucccaacccuuguaccagugcugggcucagacccugguacaggccugggggacagggaccuggggac

SEQ ID NO: 25 - RNA, 87 nt, Homo sapiens
ugucccccccggcccagguucugugauacacuccgacucgggcucuggagcagucagugcaugacagaacuugggcccggaaggacc

SEQ ID NO: 26 - RNA, 64 nt, Homo sapiens
ccccgcgacgagccccucgcacaaaccggaccugagcguuuuguucguucggcucgcgugaggc